(19) United States
(12) Patent Application Publication
(10) Pub. No.: US 2024/0325073 A1
(43) Pub. Date: Oct. 3, 2024

(54) LEAFLET GRASPING AND CUTTING DEVICE

(71) Applicant: Evalve, Inc., Santa Clara, CA (US)

(72) Inventor: Chad J. Abunassar, Alameda, CA (US)

(21) Appl. No.: 18/735,666

(22) Filed: Jun. 6, 2024

Related U.S. Application Data
(63) Continuation of application No. 17/306,412, filed on May 3, 2021, now Pat. No. 12,048,448.
(60) Provisional application No. 63/020,622, filed on May 6, 2020.

(51) Int. Cl. A61B 18/14; A61B 18/00

(52) U.S. Cl. CPC A61B 18/1445; A61B 18/1492; A61B 2018/00196; A61B 2018/00369; A61B 2018/00601; A61B 2018/1407; A61B 2018/1412; A61B 2018/1425; A61B 2018/1455

(57) ABSTRACT

A system configured to cut leaflet tissue at a cardiac valve may comprise a guide catheter and a cutting mechanism routable through the guide catheter. The cutting mechanism includes a cutting arm and a plurality of grasping arms rotatably coupled to the cutting arm. The grasping arms are connected to the cutting arm via a central hinge and are actuatable between a closed position against the cutting arm and an open position where the grasping arms extend laterally away from the cutting arm by rotating about the hinge. Targeted cardiac leaflet tissue may be grasped between the cutting arm and the grasping arms and cut by a cutting element that is attached to or extends through the cutting arm.

### CROSS-REFERENCE TO RELATED APPLICATIONS

This application is a continuation of U.S. application Ser. No. 17/306,412, filed May 3, 2021, which claims priority to U.S. Provisional Patent Application No. 63/020,662, filed May 6, 2020, the entire contents of which are incorporated by reference herein.

### BACKGROUND OF THE INVENTION

The mitral valve controls blood flow from the left atrium to the left ventricle of the heart, preventing blood from flowing backwards from the left ventricle into the left atrium so that it is instead forced through the aortic valve for delivery of oxygenated blood throughout the body. A properly functioning mitral valve opens and closes to enable blood flow in one direction. However, in some circumstances the mitral valve is unable to close properly, allowing blood to regurgitate back into the atrium.

Mitral valve regurgitation has several causes. Functional mitral valve regurgitation is characterized by structurally normal mitral valve leaflets that are nevertheless unable to properly coapt with one another to close properly due to other structural deformations of surrounding heart structures. Other causes of mitral valve regurgitation are related to defects of the mitral valve leaflets, mitral valve annulus, or other mitral valve tissues.

The most common treatments for mitral valve regurgitation rely on valve replacement or repair including leaflet and annulus remodeling, the latter generally referred to as valve annuloplasty. One technique for mitral valve repair which relies on suturing adjacent segments of the opposed valve leaflets together is referred to as the “bowtie” or “edge-to-edge” technique. While all these techniques can be effective, they usually rely on open heart surgery where the patient's chest is opened, typically via a sternotomy, and the patient placed on cardiopulmonary bypass. The need to both open the chest and place the patient on bypass is traumatic and has associated high mortality and morbidity. In some patients, a fixation device can be installed into the heart using minimally invasive techniques. The fixation device can hold the adjacent segments of the opposed valve leaflets together and may reduce mitral valve regurgitation. One such device used to clip the anterior and posterior leaflets of the mitral valve together is the MitraClip® fixation device, sold by Abbott Vascular, Santa Clara, California, USA.

However, sometimes after a fixation device is installed, undesirable mitral valve regurgitation can still exist, or can arise again. For these sub-optimally treated patients, the presence of a fixation device in their mitral valves may obstruct additional procedures such as transcatheter mitral valve replacement. These patients may also be considered too frail to tolerate open-heart surgery, so they are often left with no viable options to further improve the function of their mitral valve.

Accordingly, it would be desirable to provide alternative and additional methods, devices, and systems for removing and/or disabling fixation devices that are already installed. At least some of these objectives will be met by the inventions described below.

### BRIEF SUMMARY

The present disclosure is directed to systems, methods, and devices configured to effectively cut leaflet tissue at a cardiac valve and thereby enable removal of a cardiac valve fixation device and/or further interventional procedures involving the cardiac valve, such as implantation of a replacement valve.

In one embodiment, a cutting mechanism includes a cutting arm having a length extending along a longitudinal axis, the cutting arm including an actuatable cutting element configured to cut targeted leaflet tissue upon sufficient contact with the targeted leaflet tissue. The cutting mechanism further includes a central hinge disposed at or near a distal end of the cutting arm. One or more grasping arms are connected to the central hinge and each extend therefrom to a respective free end. The one or more grasping arms are rotatable about the central hinge so as to be selectively moveable between a closed position in which the one or more grasping arms are closed substantially against the cutting arm and an open position in which the one or more grasping arms are opened laterally away from the cutting arm. The cutting mechanism is configured to enable grasping of leaflet tissue between the cutting arm and the one or more grasping arms and to enable the cutting of grasped leaflet tissue via actuation of the cutting element.

An embodiment of a system for cutting leaflet tissue at a cardiac valve includes a guide catheter having a proximal end and a distal end, wherein the distal end of the guide catheter is steerable to a position above a cardiac valve, and a cutting mechanism. The cutting mechanism is routable through the guide catheter and configured to extend beyond the distal end of the guide catheter. The cutting mechanism is configured to enable grasping of leaflet tissue between the cutting arm and the one or more grasping arms and to enable the cutting of grasped leaflet tissue via actuation of the cutting element.

An embodiment of a method of cutting leaflet tissue at a cardiac valve within a body includes the steps of providing a system for cutting leaflet tissue, positioning the guide catheter such that the distal end of the guide catheter is positioned near a targeted cardiac valve, extending the cutting mechanism beyond the distal end of the guide catheter, grasping targeted leaflet tissue between the cutting arm and the one or more grasping arms of the cutting mechanism, and actuating the cutting element of the cutting mechanism to cut the grasped leaflet tissue. The targeted cardiac valve may have a fixation device attached to adjacent leaflets of the cardiac valve.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates a cross-sectional view of a human heart from a superior perspective, showing the mitral valve with an implanted clip fixation device for holding anterior and posterior leaflets of the mitral valve together;

FIG. 2 illustrates an exemplary delivery system that may be utilized for guiding and/or delivering a cutting mechanism to a cardiac valve;

FIG. 3 is a perspective view of an exemplary embodiment of a cutting mechanism according to the present disclosure;

FIG. 4 illustrates a cross-sectional view of a human heart from an anterior perspective showing exemplary approaches for positioning a cutting mechanism at a targeted cardiac valve for cutting leaflet tissue therein;

FIGS. 5A-5C are more detailed views of a cutting mechanism according to the present disclosure, showing actuation of grasping arms from an open position to a closed position;

FIGS. 5D-5H illustrate exemplary configurations of the cutting mechanism of FIGS. 5A-5C including different cross bar orientations;

FIGS. 51 and 5J illustrate another embodiment of a cutting mechanism having an auxiliary arm for moving the cutting element in response to actuation of a control rod;

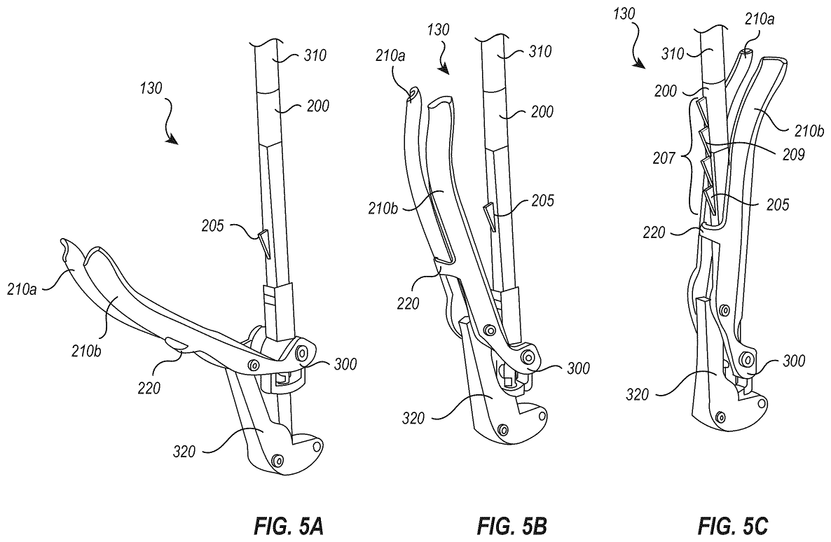

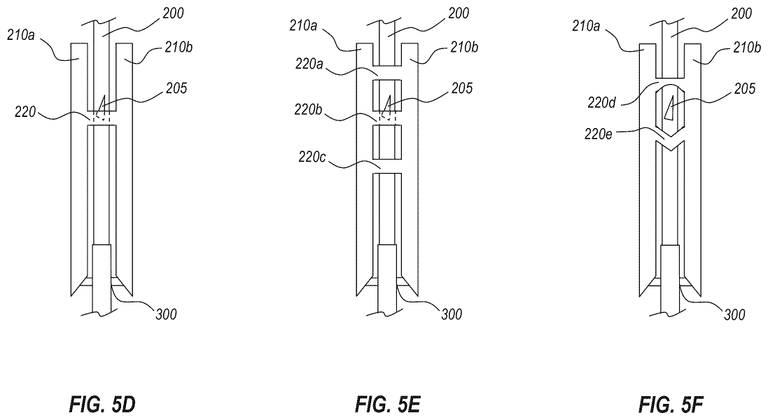

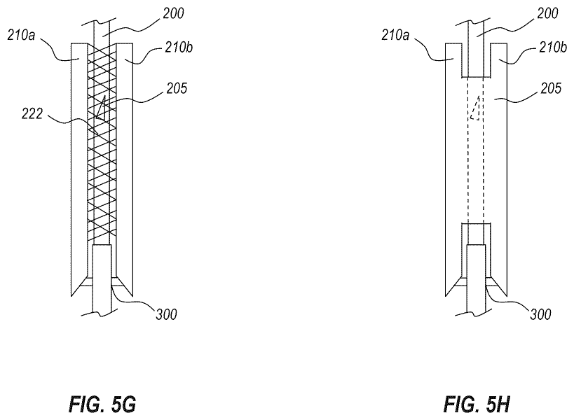

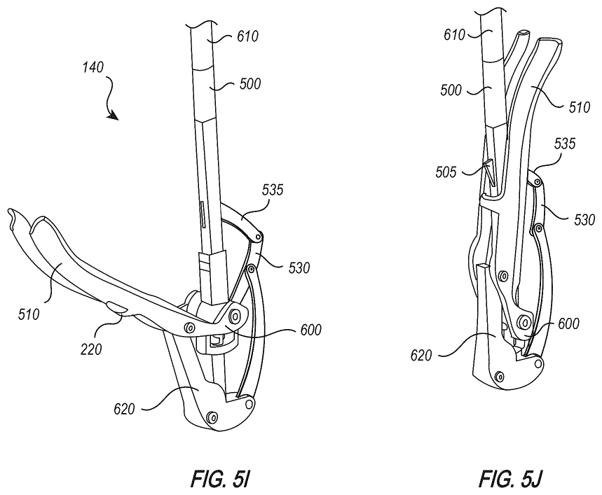

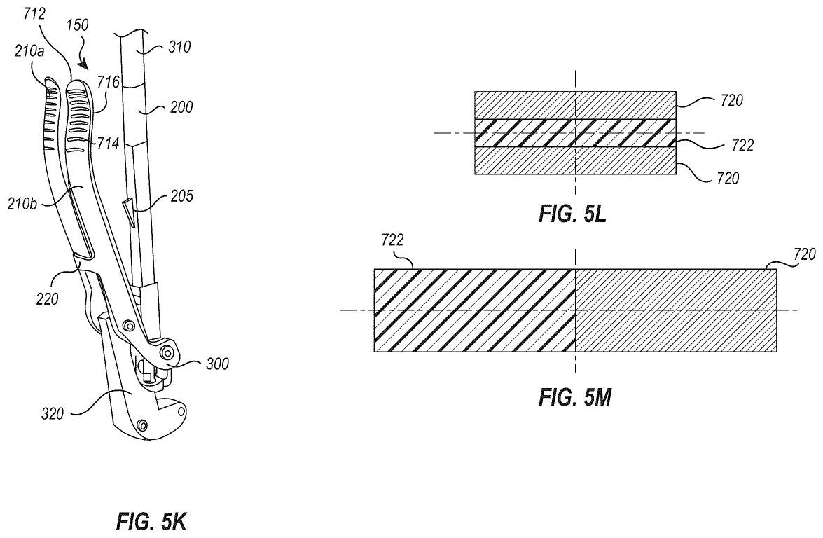

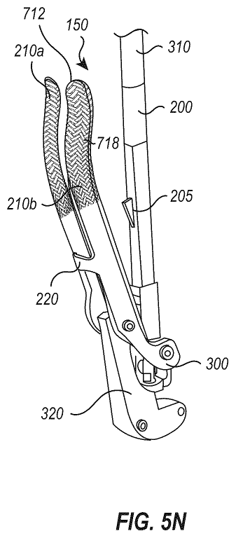

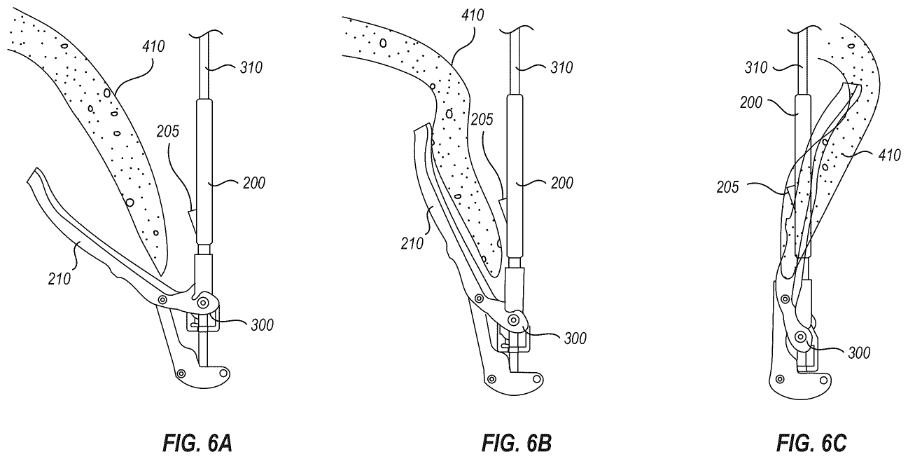

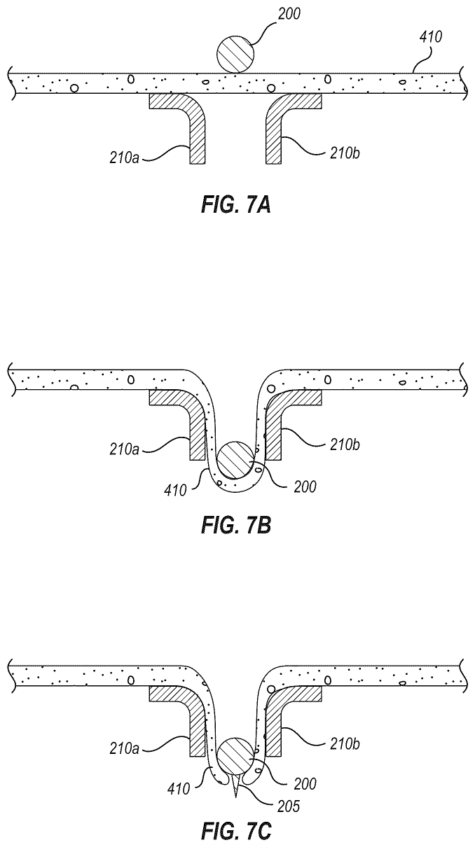

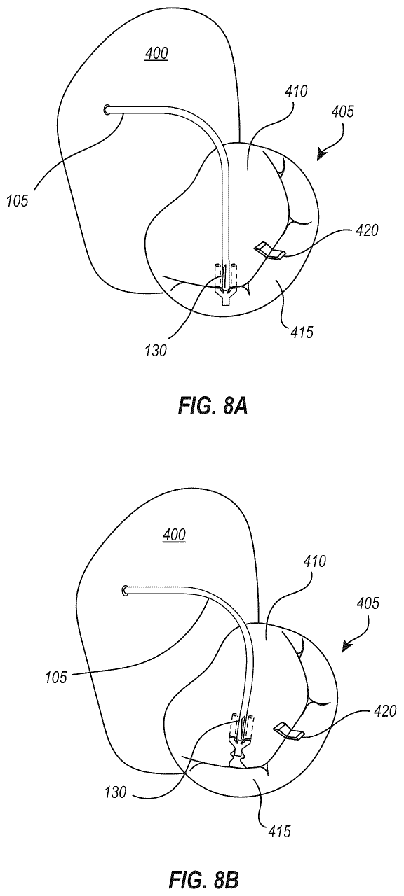

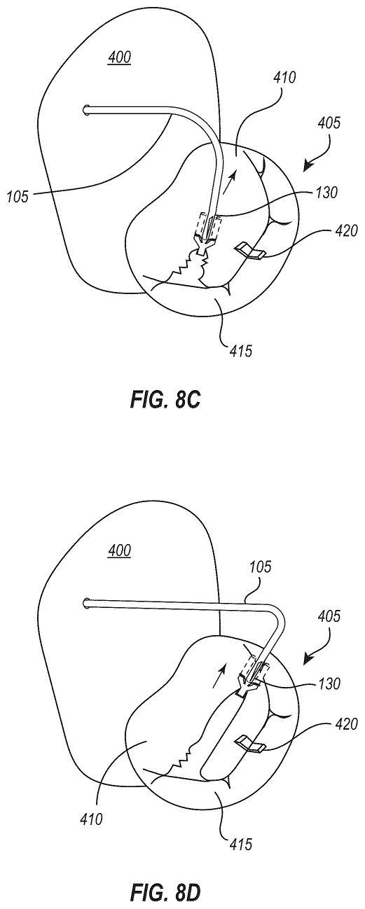

FIG. 5K illustrates another embodiment of a cutting mechanism having an auxiliary arm for moving the cutting element in response to actuation of a control rod;

FIGS. 5L and 5M illustrate alternate cross-sectional views of an auxiliary arm for moving a cutting element of the cutting mechanism;

FIG. 5N illustrates another embodiment of a cutting mechanism having an auxiliary arm for moving the cutting element in response to actuation of a control rod;

FIGS. 6A-6C illustrate the cutting mechanism of FIGS. 5A-5C while grasping leaflet tissue;

FIGS. 7A-7C illustrate a cross-sectional view of the cutting mechanism during grasping and cutting of leaflet tissue; and

FIGS. 8A-8D illustrate delivery of the cutting mechanism to the mitral valve and use of the cutting mechanism to cut targeted leaflet tissue at the mitral valve.

### DETAILED DESCRIPTION

### Introduction

Embodiments described herein are configured to effectively cut leaflet tissue at a cardiac valve and thereby enable removal of a cardiac valve fixation device and/or enable further interventional procedures involving the cardiac valve, such as implantation of a replacement valve.

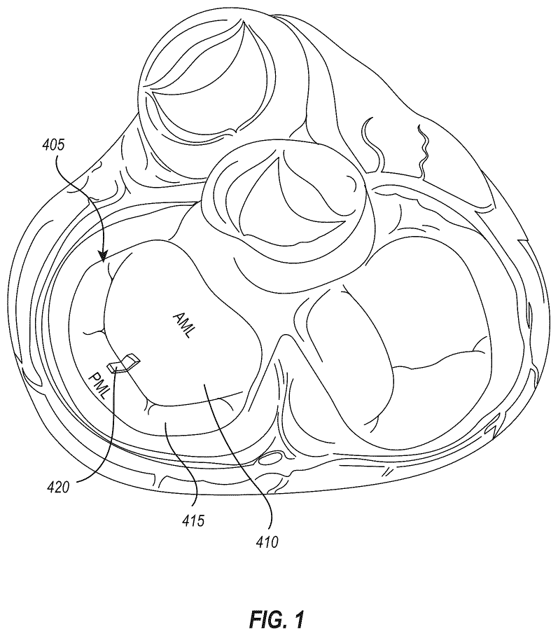

FIG. 1 illustrates a cross-sectional view of a human heart from a superior perspective, showing the mitral valve 405, which includes an anterior leaflet 410 and a posterior leaflet 415. A clip fixation device 420 has been positioned in the mitral valve 405 to clip and hold the leaflets 410 and 415 together at the coapting edges. As explained above, such repair devices are often placed with the intent of reducing mitral valve regurgitation. However, if excessive regurgitation remains following placement of the device 420, or if excessive flow obstruction develops (mitral stenosis), and further interventional procedures are necessary or desired, the fixation device 420 may need to be detached from leaflet tissue and/or removed from the mitral valve 405. For example, the fixation device 420 may need to be repositioned or removed prior to the placement of a replacement valve.

Conventional techniques for cutting leaflet tissue to remove fixation devices include using a snare wire segment energized with radiofrequency (RF) energy to cut leaflet tissue surrounding the fixation device. However, such techniques suffer from drawbacks such as difficulty in clearly visualizing the anterior leaflet 410. Typically, cutting of the anterior leaflet 410 and not the posterior leaflet 415 is desired, because cutting of the posterior leaflet 415 can result in the free fixation device 420 and anterior leaflet 410 descending into the left ventricle and obstructing the left ventricular outflow tract (LVOT).

Conventional approaches also risk snaring sub-valvular anatomic structures such as mitral valve chordae tendineae or papillary muscles. Snaring and/or cutting these structures can result in damage to ventricular health and function. Snare wires may also become caught on or in between fixation devices prior to the application of RF energy, which can result in inadvertent transmittance of RF energy to the fixation device(s) (which typically include metal components). This can result in excessive heating in the heart, leading to localized tissue damage, tissue fragmentation and embolization, and excessive coagulation.

Embodiments described herein can provide several benefits to the art. For example, embodiments described herein may allow for the clear visualization of the anterior mitral leaflet during cutting in order to avoid inadvertently cutting the posterior mitral leaflet. Embodiments described herein are also configured to provide effective tissue grasping functionality that can minimize the risk of becoming caught or entangled with sub-valvular structures or the previously implanted fixation device(s). The structures and corresponding functions that enable such benefits are described in more detail below.

Although most of the following description will focus on cutting of the anterior mitral leaflet 410, it will be understood that the same components and features may be utilized, in some applications, to additionally or alternatively target the posterior mitral leaflet 415 for cutting. Further, although most of the examples describe the application of a cut that extends laterally across the anterior leaflet 410 (see FIGS. 8A-8D), the same components and features described herein may be utilized to cut a targeted leaflet radially rather than laterally. For example, in preparation for placement of a prosthetic replacement mitral valve, the anterior leaflet 410 may be cut radially to bisect the leaflet 410 and reduce the chance of outflow tract obstruction after a prosthetic replacement valve is implanted.

Moreover, although the examples described herein are provided in the context of cutting leaflet tissue of a mitral valve, one skilled in the art will appreciate that the embodiments described herein are not necessarily limited to use within the mitral valve 405. In other applications, the targeted cardiac valve could be the tricuspid valve, aortic valve, or pulmonic valve for example. More generally, the embodiments described herein may be utilized in other implementations involving removal of a previously implanted or deployed device from tissue.

In addition, although examples may illustrate routing the guide catheter to the mitral valve via a transfemoral/transseptal or transjugular/transseptal approach, other suitable delivery approaches may be used, including radial or transapical approaches.

### Delivery System Overview

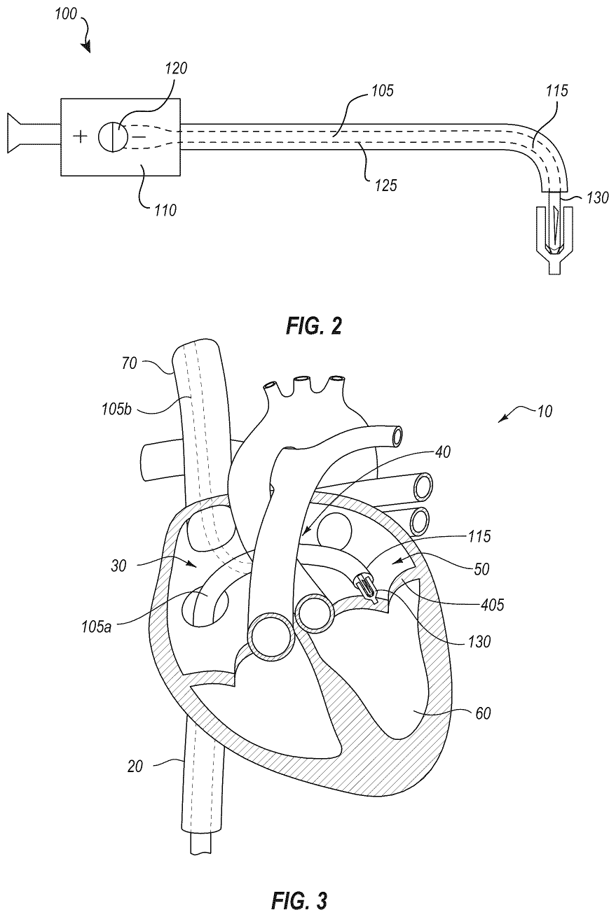

FIG. 2 illustrates an exemplary embodiment of a delivery system 100 that may be utilized for guiding and/or delivering a cutting mechanism 130 to a targeted cardiac valve. In at least one embodiment, the delivery system 100 includes a guide catheter 105 having a proximal end and a distal end 115. The delivery system may comprise a handle 110 positioned on the proximal end of the guide catheter 105. The guide catheter 105 may be operatively coupled to a handle 110. The guide catheter 105 may be steerable to enable the guiding and orienting of the guide catheter 105, including the distal end 115 of the guide catheter 105. For example, the handle 110 may include at least one control 120 (e.g., a dial, a switch, a slider, a button, etc.) that can be actuated to control the movement and curvature of the distal end 115 of the guide catheter 105.

As one example of a steering mechanism, the at least one control 120 may be operatively coupled to one or more control lines 125 (e.g., pull wires) extending from the handle 110 through the guide catheter 105 to the distal end 115 of the guide catheter (e.g., through one or more lumens in the guide catheter 105). Actuation of the at least one control 120 may adjust the tensioning of a control line 125 to pull the guide catheter 105 in the corresponding direction. FIG. 2 shows a pair of control lines 125. Alternatively, a handle 110 may comprise more than one control 120 configured for steering and any number of corresponding control lines. For example, the delivery system 100 may be configured to provide bending of the guide catheter 105 in multiple planes and/or at multiple bending points along the length of the guide catheter 105.

The control 120 and/or other controls disposed at the handle 110 may also be utilized to control actuation of various components of the cutting mechanism 130, as explained in more detail below. As shown in FIG. 2, the cutting mechanism 130 is configured in size and shape so as to be routable through the guide catheter 105 and extendable beyond the distal end 115 of the guide catheter 105. The cutting mechanism 130 may also be retracted back into the guide catheter 105. Control(s) 120 may control the cutting mechanism's 130 extension through and retraction back into the guide catheter 105. Additionally, or alternatively, the control(s) 120 may be configured to provide selective actuation of one or more components of the cutting mechanism 130.

FIG. 3 illustrates a cross-sectional view of a patient's heart 10 from an anterior perspective, showing an exemplary approach for delivering the cutting mechanism to the targeted mitral valve 405 using the guide catheter 105. In particular, FIG. 3 illustrates a transfemoral approach via guide catheter 105 (shown for this approach as guide catheter 105a), and an alternative transjugular approach via guide catheter 105 (shown for this approach as guide catheter 105b).

In a transfemoral approach, the delivery catheter 105a is inserted into the patient's vasculature at a femoral vein and directed to the inferior vena cava 20. The catheter 105a is passed through the inferior vena cava 20 and into the right atrium 30. In the transjugular approach, the delivery catheter 105b is inserted into the patient's vasculature at a jugular vein and directed to the superior vena cava 70. The catheter 105b is passed through the superior vena cava 70 and into the right atrium 30. Subsequently, in either approach, the distal end 115 of the catheter is pushed across the septum 40 so as to be positioned in the left atrium 50 superior of the mitral valve 405.

As explained further below, the cutting mechanism 130 is then directed partially through the mitral valve 405 and partially into the left ventricle 60 so that leaflet tissue can be grasped and cut.

### Cutting Mechanism Details

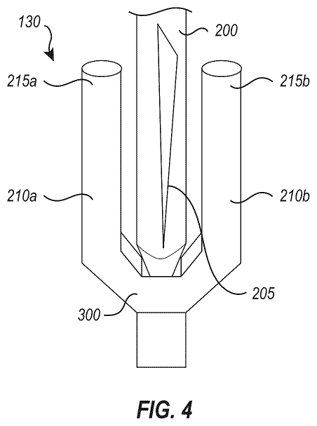

FIG. 4 is an expanded view of the cutting mechanism 130. As shown, the cutting mechanism 130 may comprise a cutting arm 200 surrounded by at least two grasping arms 210a and 210b. The cutting mechanism 130 includes a central hinge 300 disposed at the distal end of the device. The grasping arms 210a and 210b connect to the cutting arm 200 at the central hinge 300 and extend therefrom to respective free ends 215a and 215b.

The grasping arms 210a and 210b may comprise a rigid, semi-rigid, or flexible material. Preferably, at least the tips of the grasping arms 210a and 210b near the free ends 215a and 215b may comprise a flexible material so they are atraumatic if contacted against the ventricular wall or subvalvular structures.

The grasping arms 210a and 210b may have a length of about 4 to about 40 mm, more typically about 6 to about 20 mm, although smaller or longer lengths may be utilized according to particular application needs. In at least one embodiment, the length of the at least two grasping arms 210a and 210b is adjustable.

FIG. 4 further shows that the cutting mechanism 130 may comprise a cutting element 205. The cutting element 205 may be configured to cut a portion of a leaflet grasped by the cutting mechanism 130 when the cutting mechanism 130 is actuated. In at least one embodiment, the cutting element 205 is spring loaded and/or configured to retract into the cutting arm 200 when not in use.

The cutting element 205 may comprise a sharpened edge, such as a mechanical blade, as shown in FIG. 4. Additionally, or alternatively, the cutting element 205 may comprise a tapered needle, an active electrosurgical electrode blade configured to provide radio frequency current energy to the portion of the leaflet, a wire loop, or other suitable structure capable of cutting leaflet tissue grasped by the cutting mechanism 130.

The cutting element 205 may have a length of at least the majority of the length of the cutting arm 200 in order to enable more expedient cutting of the leaflets. In other embodiments, the cutting element may have a length of less than the majority of the length of the cutting arm 200, in order to provide precise cutting and reduce the risk of inadvertent cutting of incorrect tissue.

In some embodiments, the cutting element 205 is disposed within the cutting arm 200. For example, the cutting arm 200 may comprise a slot through which the cutting element passes 205. The slot may have a greater length than the cutting element 205 to allow the cutting element 205 to move proximally/distally relative to the cutting arm 200, such as in a reciprocating motion, to aid in cutting grasped leaflet tissue.

The cutting arm 200 may comprise a gripping element configured to aid in gripping leaflet tissue in contact with the cutting arm 200. For example, the cutting arm 200 may include tines, barbs, one or more coatings, grooves, textured surfaces, and/or other features for increasing the friction of the cutting arm surface to prevent grasped tissue from sliding proximally and/or away from the cutting arm 200.

FIGS. 5A-5C illustrate the exemplary cutting mechanism 130 in greater detail, showing actuation and movement of the grasping arms 210a and 210b between an open position and a closed position. FIG. 5A shows the grasping arms 210a and 210b in an open position, FIG. 5B shows the grasping arms 210a and 210b in an intermediate position, and FIG. 5C shows the grasping arms 210a and 210b in a closed position.

FIGS. 5A-5C show that the grasping arms 210a and 210b may rotate laterally away from the cutting arm 200 by way of the central hinge 300. In a typical tissue grasping maneuver, the grasping arms 210a and 210b are opened to an angle of about 30 to about 60 degrees (such as shown in FIG. 5A), though the grasping arms 210a and 210b may be configured to open to an angle of up to about 90 degrees.

The cutting mechanism 130 may include a control rod 310 that extends through the cutting arm 200 (or runs parallel with it) to mechanically couple to a linkage assembly 320 of the cutting mechanism 130. The linkage assembly 320 is in turn mechanically connected to the grasping arms 210a and 210b. The control rod 310 is able to move in the axial direction relative to the cutting arm 200. A proximal end (not shown) of the control rod 310 can extend to the handle 110 and be operatively connected to one or more controls 120 (see FIG. 2).

Actuation of the control rod 310 moves the control rod 310 axially relative to the cutting arm 200 to thereby mechanically adjust the linkage assembly 320. Because the linkage assembly 320 is connected to the grasping arms 210a and 210b, the axial movement of the control rod 310 thereby controls rotation of the grasping arms 210a and 210b towards and away from the cutting arm 200. For example, the control rod 310 and linkage assembly 320 may be configured to move the grasping arms 210a and 210b toward the cutting arm 200 (and toward the closed position) when the control rod 310 is moved proximally relative to the cutting arm 200, and to move the grasping arms 210a and 210b away from the cutting arm 200 (and toward the open position) when the control rod 310 is moved distally relative to the cutting arm 200.

Other embodiments may additionally or alternatively include other actuation mechanisms for moving the cutting mechanism 130 between an open position and a closed position. For example, the cutting mechanism 130 may be configured to move between open and closed positions based on rotation of the control rod 310 and/or based on controlling tension in one or more control wires extending from the cutting mechanism 310 to one or more controls 120 of the handle 110. In some embodiments, the cutting mechanism 130 may be biased toward a “default” position (either the open position or the closed position), and once moved away from the default, biased position, a button, toggle, switch, or other control mechanism can be actuated to trigger release and rapid movement back to the default, biased position.

The shape and length of the at least two grasping arms 210a and 210b shown in FIGS. 5A-5C are merely exemplary. The at least two grasping arms 210a and 210b may be sized and shape to increase mechanical advantage of the at least two grasping arms 210a and 210b to exert tension on leaflet tissue grasped therebetween. In at least one embodiment the grasping arms 210a and 210b are configured to shield the cutting element 205 from surrounding cardiac structures. For example, a cover, webbing, or other protective structure may be positioned over the space between the grasping arms 210a and 210b to minimize the risk of the cutting element 205 inadvertently contacting anything not grasped between the cutting arm 200 and the grasping arms 210a and 210b.

As shown by the illustrated embodiment, the grasping arms 210a and 210b may have a curved profile to better position grasped tissue for cutting. For example, the free ends 215a and 215b may be curved inward toward the cutting arm 200. The free ends 215a and 215b may curve inwards at an angle of about 5 to about 20 degrees, for example,

As shown in FIG. 5C, the grasping arms 210a and 210b may cross over the longitudinal axis of the cutting arm 200 as the grasping arms 210a and 210b move into the closed position, and this may be aided in part by the curved shape of the free ends 215a and 215b. This allows the cutting mechanism 130 to “over close” and better grasp and stretch leaflet tissue for more effective cutting of the leaflet tissue.

The shape of the cutting element 205 may be customized based on a patient's leaflet anatomy and pathology. Additionally, or alternatively, a cross bar 220 may be included partially connecting the two grasping arms 210a and 210b. The cross bar 220 may function to enhance tissue contact with the cutting arm 200 and/or cutting element 205, for example. As shown, the cross bar 220 may be disposed so as to be distal of the cutting element 205 (i.e., closer to the central hinge 300 than the cutting element 205) when the cutting mechanism 130 is in the closed position.

FIG. 5C illustrates an alternative configuration of cutting element 205, which includes a series of consecutive cutting elements 205 configured on the cutting arm 200. This configuration includes multiple cutting elements 205 can create a “serrated” blade 207, which can improve cutting performance while allowing the user to exert less than normal force during cutting. Normal force may be understood as the force used to make an adequate cut using a single cutting element 205. The dynamics of a beating heart can assist in accomplishing a full and clean cut against blade 207, which can be useful for removing stubborn pieces of leaflet tissue which might remain after an initial cutting attempt. If a strand of tissue remains, the beating of the heart can cause the strand of tissue to move or drag across the blade 27 in a dynamic way once its neighboring tissue is cut, which can subsequently aid in completion of the cut and removal of the strand of tissue. In some embodiments, all exposed cutting element edges 209 can be sharp to ensure bi-directional serrated cutting Alternatively, portions of the exposed cutting element edges 209 can be sharp.

The mitral and tricuspid valve leaflet tissue generally varies in thickness from ˜0.4 mm thick for generally healthy tricuspid leaflet tissue, to ˜3.2 mm for thick healthy mitral valve leaflet tissue. Diseased tissue may be thicker than healthy leaflet tissue as diseased tissue can be thickened to 5.0 mm and greater due to, for example, degenerative valve leaflet tissue or Barlow's disease. In some configurations, the cutting element 205 can be configured to cut to a depth sufficient to adequately cut through diseased tissue. A depth of the cutting element 205 can range from about 0.4 mm to about 10 mm, from about 0.5 mm to about 8 mm, from about 0.6 mm to about 4 mm, from about 0.8 mm to about 3 mm, from about 0.6 mm to about 10 mm, from about 0.8 mm to about 8 mm, from about 1 mm to about 4 mm, from about 1.2 mm to about 3 mm, and any combinations or modifications thereof. The cutting depth can be sufficient to cut through diseased tissue, while also shallow enough to avoid inadvertently cutting through tissue which should not be cut.

FIGS. 5D through 5H illustrate alternative configurations of the cross bar 220, grasping arms 210a, 210b, and the cutting element 205 that may be provided in addition to or as an alternative to the configuration shown in FIGS. 5A-5C. FIG. 5D illustrates that the cross bar 220 may be positioned so as to substantially align with the cutting element 205 when the cutting mechanism 130 is moved to the closed position. That is, the cross bar 220 may be positioned at the same location/height as the cutting element 205 so as to directly press tissue against the cutting element 205 when the cutting mechanism 130 moves into the closed position.

FIG. 5E illustrates that multiple cross bars 220 may be included (shown here as separate cross bars 220a through 220c). It will be appreciated that the cross bar(s) may be disposed at any of the locations along the lengths of the grasping arms 210a, 210b relative to the cutting element 205. For example, one or more cross bars 220a may be positioned above the cutting element 205 (i.e., farther from the central hinge 300 than the cutting element 205), one or more cross bars 220b may be substantially aligned with the cutting element 205, and/or one or more cross bars 220c may be positioned below the cutting element 205 (i.e., closer to the central hinge 200 than the cutting element 205).

FIG. 5F illustrates that cross bars 220 need not necessarily be linearly disposed between the grasping arms 210a, 210b and may have, for example, a curved shape (such as cross bar 220d), angular shape (such as cross bar 220e), shape of variable width, or other non-linear and/or variable shape. FIG. 5G illustrates a configuration that includes a webbing 222 disposed between the grasping arms 210a and 210b. The webbing 222 may comprise a braided material, sheet, and/or film, for example, that extends across the gap between the grasping arms 210a and 210b. The webbing 222 may be formed to be cut-resistant or may be allowed to be cut along with tissue when the cutting element 205 is actuated. FIG. 5H illustrates a configuration where the gap between the grasping arms 210a and 210b is substantially filled so as to form a single, “solid” grasping arm structure. The grasping arm structure may have one or more grooves or indentations for receiving the cutting arm 200 and cutting element 205.

FIGS. 51 and 5J illustrates another embodiment of a cutting mechanism 140. The cutting mechanism 140, like the cutting mechanism 130 described above, may include a cutting arm 500, a cutting element 505, one or more grasping arms 510, a central hinge 600, a control rod 610, and a linkage assembly 620.

The cutting mechanism 140 additionally includes an auxiliary arm 530 disposed opposite the one or more grasping arms 510. The auxiliary arm 530 may be connected to the linkage assembly 620 and be configured to rotate about the central hinge 600 in response to controlled actuation via the control rod 610 in a fashion similar to the grasping arms 510. The auxiliary arm 530 includes a cutting element link 535 that connects to the cutting element 505. For example, the link 535 may extend through the cutting arm 500 to connect to the cutting element 505.

The auxiliary arm 530 can thus function to move the cutting element 505 in response to actuation (via axial movement) of the control rod 610. For example, actuation of the control rod 610 can cause the grasping arms 510 and the auxiliary arm 530 to move toward the closed position. As the auxiliary arm 530 closes and gets closer to the cutting arm 500, the link 535 pushes against the cutting element 505 and causes it to extend out from the cutting arm 500, as shown in FIG. 5E. Similarly, moving the grasping arms 510 and auxiliary arm 530 to the open position causes the cutting blade 505 to retract toward the cutting arm 500.

In use, the illustrated embodiment beneficially enables the cutting element 505 to be housed or substantially housed within the cutting arm 500 when the cutting mechanism 140 is in the open position, and enables the cutting element 505 to automatically extend and be exposed only as the grasping arms 510 are closing and targeted tissue is being brought into the cutting arm 500 to be cut.

As with the cutting element 205, the cutting element 505 can extend out from the cutting arm 500 a distance ranging from about 0.4 mm to about 10 mm, from about 0.5 mm to about 8 mm, from about 0.6 mm to about 4 mm, from about 0.8 mm to about 3 mm, from about 0.6 mm to about 10 mm, from about 0.8 mm to about 8 mm, from about 1 mm to about 4 mm, from about 1.2 mm to about 3 mm, and any combinations or modifications thereof. The cutting depth, or the distance which the cutting element 505 extends from the cutting arm 500 can be sufficient to cut through diseased tissue, while also shallow enough to avoid inadvertently cutting through tissue which should not be cut.

FIG. 5K illustrates another embodiment of a cutting element 150. The cutting mechanism 150, like the cutting mechanism 130 described above, may include a cutting arm 200, a cutting element 205, one or more grasping arms 210, a central hinge 300, a control rod 310, and a linkage assembly 320.

The cutting mechanism 150 additionally includes trauma reducing features. The grasping arms 210 which are used to press leaflet tissue into the cutting element 205 might become inadvertently caught on the patient's anatomy, so it may be useful to include trauma reducing features such as atraumatic tips 712 and flexibility-enhancing features, such as slit cuts 714. Other flexibility-enhancing features may include the grasping arms 210 having a tapered cross-section, and/or the grasping arms 210 may comprise multiple materials, with a more flexible material disposed towards the distal ends 716 of the grasping arms 210 to increase flexibility toward the distal ends 716 of the grasping arms 210. The multiple materials can include at least two metallic materials, at least two polymeric materials, at least one metallic material with at least one polymeric material, combinations or modifications thereof. For instance, one material can be over molded with another material. In another configuration, one material is bonded, glued, welded, brazed with or otherwise attached to another material.

The grasping arms 210 may still maintain their stiffness near the cutting element 205 to maintain their effectiveness, though by configuring the distal ends 716 of the grasping arms 210 to bend during use, damage to the leaflet tissue or chords can be minimized. The slit cuts 714 may be equally spaced along the distal ends 716 to impart an equal degree of flexibility along the distal ends 716. The slit cuts 714 may also be arranged in a pattern where the number and density of slit cuts 714 can increase towards the distal ends 716 to provide an increasing degree of flexibility towards the atraumatic tips 712. The pattern can include discrete or overlapping slits that are orientated parallel, perpendicular, and/or transverse to a longitudinal axis of a grasping arm 210.

The grasping arms 210 can comprise a metallic material such as steel, cobalt, chrome, NITINOL®, titanium, or the like, or a polymeric material, such as poly-L-lactide (PLLA), poly (lactic-co-glycolic acid) (PLGA), Polyether block amide (PEBA), such as PEBAX®, biocompatible composite, combinations and/or modifications thereof, or the like, or the grasping arms 210 can be a combination of a metallic material and a polymeric material. FIGS. 5L and 5M illustrate examples of various configurations of materials comprising the grasping arms 210 comprising material A 720 and material B 722. In some examples, material A 720 can comprise a metallic material or a polymeric material, as discussed above. Accordingly, material B 722 can comprise a metallic material or a polymeric material. Material A 720 and material B 722 are different materials and can be configured in a sandwich configuration as shown in FIG. 5L, or they can be arranged next to each other as shown in FIG. 5M. The materials can be arranged in a manner that imparts a desired stiffness and/or flexibility on the grasping arms 210.

All or a portion of each of the grasping arms 210 can also or alternatively have a braided structure 718, as illustrated in FIG. 5N, and may be laser cut from a tube or a sheet, or made from stamped or formed material. The braided structure 718 can have a larger, looser braid at the tips 712 in order to impart a desired degree of flexibility. The braided structure 718 can become progressively tighter woven and stiffer as the braided structure 718 progresses towards the middle of the grasping arms 210. The geometry of the grasping arms 210 as shown in FIGS. 5A-5K and 5N is curved with an inward bias towards the cutting arm 200, however, in some embodiments, the profile of the grasping arms 210 may be curved with an outward bias and flare outward relative to the cutting arm 200 in order to match the curvature of the leaflet tissue being cut.

### Tissue Grasping & Cutting

FIGS. 6A-6C illustrate additional views of the cutting mechanism 130 to show use of the cutting mechanism 130 to grasp and cut leaflet tissue (such as the anterior leaflet 410 shown here). Upon routing the cutting mechanism 130 to the appropriate position at the mitral valve, the cutting mechanism 130 may be moved from the closed position to the open position. Typically, the cutting mechanism 130 is positioned so that the grasping arms 210 are on the ventricular side of the valve, and then the cutting mechanism 130 is actuated to move the grasping arms 210 to the open position. As described above, the grasping arms 210 may be actuated by moving the control rod 310 axially relative to the cutting arm 200 to cause the linkage assembly 320 to rotate the grasping arms 210 outward.

FIG. 6A shows the cutting mechanism 130 in the open position (e.g., with the grasping arms 210 open to an angle of about 60 degrees) with the grasping arms 210 on the ventricular side of the leaflet 410. The cutting mechanism 130 may be moved to appropriately position the targeted leaflet 410 between the grasping arms 210 and the cutting arm 200. Then, as shown in FIG. 6B, the grasping arms 210 may be moved toward the closed position by axially moving the control rod 310 relative to the cutting arm 200. Further actuation leads to further closing of the grasping arms 210 until the leaflet 410 is brought into contact with the cutting element 205, as shown in FIG. 6C.

In some embodiments, the cutting element 205 is connected to the control rod 310, and the control rod 310 extends through the cutting arm 200. The cutting element 205 may pass through a slit in the cutting arm 200, for example. In such an embodiment, axial movement of the control rod 310 causes corresponding axial movement of the cutting element 205 relative to the cutting arm 200. This beneficially provides an axial cutting motion of the cutting element 205 while the grasping arms 210 are moving.

For example, moving the control rod 310 proximally may simultaneously close the grasping arms 210 and cause the cutting element 205 to move proximally, allowing for an effective cutting motion that simultaneously brings the leaflet 410 laterally into the cutting element 205 while axially moving the cutting element 205 to cut the leaflet 410. This can also be utilized to perform a “reciprocating cut” procedure where the cutting element 205 reciprocates axially while the grasping arms 210 are successively opened and closed to grasp new areas of leaflet tissue.

FIGS. 7A-7C illustrate the cutting element 130 while grasping and cutting leaflet tissue from a cross-sectional view across the longitudinal axis of the cutting mechanism. FIGS. 7A-7C beneficially illustrate how the cutting element 130 may be utilized to stretch the grasped leaflet tissue (such as the anterior leaflet 410) across the cutting arm 200 for effective cutting of the tissue by the cutting element 205.

FIG. 7A shows the leaflet 410 disposed between the cutting arm 200 and the grasping arms 210a and 210b as the device approaches the closed position. FIG. 7B illustrates further closing of the grasping arms 210a and 210b such that the grasping arms 210a and 210b cross the longitudinal axis of the cutting arm 200 in an “over closed” fashion. This serves to tighten and stretch the grasped tissue across the cutting side of the cutting arm 200 so that it can be effectively cut by contact with the cutting element 205, as shown in FIG. 7C.

FIGS. 7A-7C also illustrate that the grasping arms 210a and 210b may have a curved or flared cross-sectional shape. Such a shape may correspond beneficially with the shape of the cutting arm 200 and may aid in providing the tightening and/or stretching of the grasped leaflet tissue across the cutting arm 200.

FIGS. 8A-8D are perspective views of the exemplary cutting mechanism 130 as used to cut leaflet tissue of the mitral valve 405. An interventional fixation device 420 creates a first and second orifice between the anterior mitral leaflet 410 and the posterior mitral leaflet 415 by approximating the adjacent leaflets 410 and 415. As shown, the distal end 115 of the guide catheter 105 has been extended through the septum 400 of the heart. The cutting mechanism 130 may be routed through the guide catheter 105 so as to extend through an orifice of the mitral valve 405 and be at least partially disposed on a ventricular side of the valve 405.

The grasping arms may then be actuated to move the cutting mechanism 130 to the open position. The cutting mechanism 130 is then positioned so that leaflet tissue, such as tissue of the anterior leaflet 410, resides between the grasping arms and the cutting arm. The cutting mechanism 130 is then moved to the closed position to grasp the leaflet tissue between the cutting arm and the grasping arms. The leaflet tissue may be secured by the grasping arms on a ventricular side of the mitral valve 405 and by the cutting arm on the atrial side of the mitral valve 405. With the application of some clamping force, the grasping arms can stretch the leaflet tissue across the cutting arm thereby reducing the thickness of the secured leaflet tissue at that location, and thereby better enable the cutting element to cut through the entire thickness of the secured leaflet tissue.

As shown in FIG. 4C, the cutting mechanism 130 may be advanced through the anterior mitral leaflet 410 by repeated repositioning of the cutting mechanism 130 and repeated actuation of the grasping arms. Additionally, or alternatively, the cutting mechanism 130 may be advanced through the anterior leaflet 410 by partially actuating the at least two grasping arms (e.g., to an open angle of about 15 to about 45 degrees), dynamically positioning the cutting mechanism 130, and moving or dragging the cutting mechanism 130 along the anterior mitral leaflet 410.

### Additional Exemplary Embodiments

Following are some further example embodiments of the invention. These are presented only by way of example and are not intended to limit the scope of the invention in any way.

Embodiment 1. A cutting mechanism for cutting leaflet tissue at a cardiac valve, the cutting mechanism comprising: a cutting arm having a length extending along a longitudinal axis, the cutting arm including an actuatable cutting element configured to cut targeted leaflet tissue upon sufficient contact with the targeted leaflet tissue; a central hinge disposed at or near a distal end of the cutting arm; one or more grasping arms each connected to the central hinge and extending therefrom to a respective free end, the one or more grasping arms being rotatable about the central hinge so as to be selectively moveable between a closed position wherein the one or more grasping arms are closed substantially against the cutting arm, and an open position wherein the one or more grasping arms are opened laterally away from the cutting arm by rotating about the central hinge, wherein the cutting mechanism is configured to enable grasping of leaflet tissue between the cutting arm and the one or more grasping arms and to enable the cutting of grasped leaflet tissue via actuation of the cutting element.

Embodiment 2. The cutting mechanism of claim 1, wherein the cutting mechanism comprises at least two grasping arms disposed such that, when the cutting mechanism is in the closed position, a first grasping arm is disposed on a first side of the cutting arm and a second grasping arm is disposed on a second, opposite side of the cutting arm.

Embodiment 3. The cutting mechanism of Embodiment 2, wherein the cutting element is disposed on a portion of the cutting arm between the first and second grasping arms so as to enable cutting of leaflet tissue disposed between the first and second grasping arms.

Embodiment 4. The cutting mechanism of Embodiment 2 or Embodiment 3, further comprising a cross bar extending between the first and second grasping arms.

Embodiment 5. The cutting mechanism of Embodiment 4, wherein the cross bar is aligned with the cutting element when the cutting mechanism is in the closed position, or is positioned closer to the central hinge than the cutting element when the cutting mechanism is in the closed position.

Embodiment 6. The cutting mechanism of any one of Embodiments 1-5, wherein the one or more grasping arms are openable to an angle of up to about 90 degrees from the cutting arm.

Embodiment 7. The cutting mechanism of any one of Embodiments 1-6, wherein the free ends of the one or more grasping arms curve laterally inward toward the cutting arm.

Embodiment 8. The cutting mechanism of Embodiment 7, wherein the curved free ends of the one or more grasping arms cross over the longitudinal axis of the cutting arm when moving from the open position to the closed position.

Embodiment 9. The cutting mechanism of Embodiment 7 or Embodiment 8, wherein the curved free ends curve laterally inward at an angle of between about 5 degrees and about 20 degrees.

Embodiment 10. The cutting mechanism of any one of Embodiments 1-9, wherein the cutting element is spring-loaded so as to be capable of actuation from a non-deployed to a deployed state via controlled release of spring energy.

Embodiment 11. The cutting mechanism of any one of Embodiments 1-10, wherein the central hinge comprises a linkage mechanism configured to convert axial movement of a control rod along the longitudinal axis of the cutting arm into lateral movement of the grasping arms.

Embodiment 12. The cutting mechanism of Embodiment 11, wherein the cutting element is connected to the control rod and extends through a slot of the cutting arm such that actuation of the control rod moves the cutting element within the slot in an axial direction relative to the cutting arm.

Embodiment 13. The cutting mechanism of any one of Embodiments 1-12, wherein the one or more grasping arms have a length of about 4 mm to about 40 mm.

Embodiment 14. The cutting mechanism of any one of Embodiments 1-13, wherein the cutting element comprises a sharpened edge.

Embodiment 15. The cutting mechanism of any one of Embodiments 1-14, wherein the cutting element comprises a tapered needle.

Embodiment 16. The cutting element of any one of Embodiments 1-15, wherein the cutting element comprises a wire loop configured to provide radio frequency current energy.

Embodiment 17. The cutting element of any one of Embodiments 1-16, wherein the cutting arm includes a gripping element configured to aid in gripping leaflet tissue in contact with the cutting arm.

Embodiment 18. The cutting mechanism of any one of Embodiments 1-17, further comprising an auxiliary arm connected to the central hinge and disposed opposite the one or more grasping arms, the auxiliary arm being mechanically connected to the cutting element such that movement of the auxiliary arm as the cutting mechanism moves between the open position and the closed position causes advancement and retraction of the cutting element relative to the cutting arm.

Embodiment 19. The cutting mechanism of any of the Embodiments 1-18, wherein the cutting element comprises at least two or more cutting elements.

Embodiment 20. The cutting mechanism of any of the Embodiments 1-19, wherein the cutting element has a depth of about 0.4 mm to about 10 mm.

Embodiment 21. A system for cutting leaflet tissue at a cardiac valve, the system comprising: a guide catheter having a proximal end and a distal end, wherein the distal end of the guide catheter is steerable to a position above a cardiac valve; and a cutting mechanism as in any one of Embodiments 1-20 routable through the guide catheter and configured to extend beyond the distal end of the guide catheter.

Embodiment 22. The system of Embodiment 21, further comprising a handle coupled to the proximal end of the guide catheter, the handle comprising at least one control operatively connected to the cutting mechanism to enable actuation of the grasping arms between the closed position and the open position and/or to enable actuation of the cutting element.

Embodiment 23. A method of cutting leaflet tissue at a cardiac valve within a body, the method comprising: providing a system for cutting leaflet tissue as in any of the Embodiment 1-22, positioning the guide catheter such that the distal end of the guide catheter is positioned near a targeted cardiac valve; extending the cutting mechanism beyond the distal end of the guide catheter; grasping targeted leaflet tissue between the cutting arm and the one or more grasping arms of the cutting mechanism; and actuating the cutting element of the cutting mechanism to cut the grasped leaflet tissue.

Embodiment 24. The method of Embodiment 23, wherein the one or more grasping arms are positioned on a ventricular side of the targeted leaflet tissue and the cutting arm is positioned on an atrial side of the targeted leaflet tissue when the leaflet tissue is grasped by the cutting mechanism.

Embodiment 25. The method of Embodiment 23 or Embodiment 24, wherein the cutting mechanism is advanced through the leaflet by repeated actuation of the one or more grasping arms between the open and closed positions so as to grasp and cut successive portions of leaflet tissue.

Embodiment 26. The method of any one of Embodiments 23-25, wherein the cutting mechanism is advanced through the leaflet tissue by positioning the one or more grasping arms to a position between a fully closed and fully open position, and moving the cutting mechanism through the leaflet tissue.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

We claim:

1. A cutting mechanism configured for severing leaflet tissue at a cardiac valve, the cutting mechanism comprising:
a cutting arm having a length and including an actuatable cutting element configured to sever targeted leaflet tissue;
a central hinge disposed distal with respect to the cutting element;
a grasping arm connected to the central hinge and extending therefrom to a free end, the grasping arm being rotatable about the central hinge so as to be selectively moveable between a closed position and an open position to vary an angle between the grasping arm and the cutting arm,
wherein the angle is greater in the open position as compared to the closed position,
wherein in the closed position, the grasping arm is positioned at least partially against the cutting arm, and
further wherein the cutting mechanism is configured to enable grasping of leaflet tissue between the cutting arm and the grasping arm and to enable the severing of grasped leaflet tissue via actuation of the cutting element.

2. The cutting mechanism of claim 1, wherein the central hinge comprises a linkage assembly configured to convert axial movement of a control rod along a longitudinal axis of the cutting arm into lateral movement of the grasping arm.

3. The cutting mechanism of claim 2, wherein the cutting element is connected to the control rod and extends through a slot of the cutting arm such that actuation of the control rod moves the cutting element within the slot in an axial direction relative to the cutting arm.

4. The cutting mechanism of claim 1, wherein the cutting element is selected from a group comprising a sharpened edge, a tapered needle, and a wire loop configured to provide radio frequency current energy.

5. The cutting mechanism of claim 1, wherein the grasping arm has an opening therein extending from the free end of the grasping arm toward the central hinge, the opening in the grasping arm being disposed such that when the cutting mechanism is in the closed position a first portion of the grasping arm is disposed on a first side of the cutting arm and a second portion of the grasping arm is disposed on a second, opposite side of the cutting arm.

6. The cutting mechanism of claim 1, wherein the grasping arm of the cutting mechanism comprises a first grasping arm and a second grasping arm, the first grasping arm and the second grasping arm being disposed such that when the cutting mechanism is in the closed position, the first grasping arm is disposed on a first side of the cutting arm and a second grasping arm is disposed on a second, opposite side of the cutting arm.

7. The cutting mechanism of claim 6, wherein the cutting element is disposed on a portion of the cutting arm between the first and second grasping arms to enable cutting of leaflet tissue disposed between the first and second grasping arms.

8. The cutting mechanism of claim 6, further comprising a cross bar extending between the first and second grasping arms.

9. The cutting mechanism of claim 1, wherein the cutting element is spring-loaded so as to be capable of actuation from a non-deployed to a deployed state via controlled release of spring energy.

10. A system for severing leaflet tissue at a cardiac valve, the system comprising:
a guide catheter having a proximal end and a distal end, the distal end of the guide catheter being configured to be steerable to a position at a cardiac valve; and
a handle coupled to the proximal end of the guide catheter, the handle comprising a control operatively connected to a cutting mechanism;
the cutting mechanism is routable through the guide catheter and is configured to extend beyond the distal end of the guide catheter, the cutting mechanism comprising:
a cutting arm having a length extending along a longitudinal axis, the cutting arm including an actuatable cutting element configured to severe targeted leaflet tissue upon sufficient contact with the targeted leaflet tissue;
a central hinge disposed distal with respect to the cutting element of the cutting arm;
a grasping arm connected to the central hinge and extending therefrom to a respective free end, the grasping arm being rotatable about the central hinge so as to be selectively moveable between a closed position in which the grasping arm is at least partially positioned against the cutting arm, and an open position, wherein an angle formed between the grasping arm and the cutting arm at the central hinge is greater in the open position as compared to the closed position,
wherein the cutting mechanism is configured to enable grasping of leaflet tissue between the cutting arm and the grasping arm and to enable the severing of grasped leaflet tissue via actuation of the cutting element.

11. The system of claim 10, wherein the control is operatively connected to the cutting mechanism to enable actuation of the grasping arm between the closed position and the open position and/or to enable actuation of the cutting element.

12. The system of claim 10, further comprising an auxiliary arm connected to the central hinge and disposed opposite the grasping arm, the auxiliary arm being mechanically connected to the cutting element such that movement of the auxiliary arm as the cutting mechanism moves between the open position and the closed position causes advancement and retraction of the cutting element relative to the cutting arm.

13. The system of claim 10, wherein the cutting element is spring-loaded so as to be capable of actuation from a non-deployed to a deployed state via the at least one control in the handle.

14. The system of claim 10, wherein the grasping arm of the cutting mechanism comprises a first grasping arm and a second grasping arm, the first grasping arm and the second grasping arm being disposed such that when the cutting mechanism is in the closed position, the first grasping arm is disposed on a first side of the cutting arm and the second grasping arm is disposed on a second, opposite side of the cutting arm.

15. The system of claim 14, wherein the cutting mechanism further comprises a cross bar extending between the first grasping arm and the second grasping arm, the cross bar being aligned with the cutting element when the cutting mechanism is in the closed position.

16. The system of claim 14, wherein the cutting mechanism further comprises a cross bar extending between the first grasping arm and the second grasping arm, the cross bar being positioned closer to the central hinge than the cutting element when the cutting mechanism is in the closed position.

17. A method of severing leaflet tissue at a cardiac valve within a body, the method comprising:
providing a system for severing leaflet tissue, the system comprising:
a guide catheter having a proximal end and a distal end; and
a cutting mechanism comprising:
a cutting arm having a length extending along a longitudinal axis, the cutting arm including an actuatable cutting element;
a central hinge disposed distal with respect to the cutting arm; and
a grasping arm connected to the central hinge and extending therefrom to a free end, the grasping arm being rotatable about the central hinge so as to be selectively moveable;
positioning the guide catheter such that the distal end of the guide catheter is positioned near a targeted cardiac valve;
extending the cutting mechanism beyond the distal end of the guide catheter; and
actuating the cutting element of the cutting mechanism to severe the grasped leaflet tissue.

18. The method of claim 17, wherein the grasping arm is positioned on a ventricular side of the targeted leaflet tissue and the cutting arm is positioned on an atrial side of the targeted leaflet tissue when the leaflet tissue is grasped by the cutting mechanism.

19. The method of claim 17, wherein the cutting mechanism is advanced at least partially through the leaflet by actuation of the grasping arm between open and closed positions to grasp and cut the leaflet tissue.

20. The method of claim 17, wherein the system includes a plurality of grasping arms, wherein one or more grasping arms are positioned on a ventricular side of the targeted leaflet tissue and the cutting arm is positioned on an atrial side of the targeted leaflet tissue when the leaflet tissue is grasped by the cutting mechanism.
